(19) United States
(12) Patent Application Publication
(10) Pub. No.: US 2004/0175811 A1
(43) Pub. Date: Sep. 9, 2004

(54) Method for fixing biomolecules onto chemically inert surfaces

(76) Inventors: Hans-Willi Kling, Wuppetal (DE); Jens Seemann, Erkrath (DE); Johannes Georg Schindler, Marburg/Lahn (DE)

Correspondence Address: COGNIS CORPORATION, PATENT DEPARTMENT, 300 BROOKSIDE AVENUE, AMBLER, PA, 19002, US

(21) Appl. No.: 10/474,769

(22) PCT Filed: Apr. 11, 2002
(86) PCT No.: PCT/EP02/04042

(30) Foreign Application Priority Data
Apr. 14, 2001 (DE) 10118553.7
Apr. 14, 2001 (DE) DE10118554.5

(51) Int. Cl. C12N 11/06; C12N 11/00

(52) U.S. Cl. 435/174; 435/181

(57) ABSTRACT

Biomolecules such as enzymes can be immobilized on a surface by a process which comprises the steps of: (1) fixing a compound having a functional group selected from the group consisting of a carboxyl group, an amino group, a hydroxy group, a thio group and a combination thereof to a surface by plasmachemical modification of the surface to form a fixed surface; (2) binding a biomolecule to the fixed surface by reaction of the biomolecule with one or more of the functional groups.

This invention relates to a process for binding biomolecules, more particularly enzymes or enzymatic systems, to chemically inert carrier surfaces. More particularly, the present invention relates to a process for immobilizing biomolecules, more particularly enzymes or enzymatic systems, by binding, more particularly physical and/or chemical binding, to chemically inert carrier surfaces and to the use of the systems thus immobilized, preferably in bioreactors, biosensors and chromatographic systems.

In applied microbiology, more particularly in biotechnology, it is known that enzymes, enzyme-producing microorganisms or cells can be fixed to certain carriers, particularly if they are used as biocatalysts. This process is known generally as immobilization.

Since native enzymes are reduced in their activity by biological, chemical or physical effects during storage or in “one-off” batch applications, there is a need to stabilize the enzymes in view of their high production costs. Through immobilization, the enzymes become reusable. After use, the enzymes are easy to remove. In this way, they can be used in high local concentrations and in continuous throughflow. The substrate specificity and the specificity of the reaction and also the reactivity of the enzymes should not be lost as a result of immobilization.

In general, enzymes can be immobilized by three basic methods, namely: first, immobilization by crosslinking, second immobilization by binding to a carrier and, third, immobilization by enclosure.

Where immobilization is carried out by crosslinking, the crosslinked enzymes obtained are fixed to one another without any effect on their activity. However, the enzymes are no longer soluble. Crosslinking is carried out, for example, with glutardialdehyde.

Where enzymes are immobilized by binding to a carrier, binding may be carried out by adsorption, ionic bonding or covalent bonding. Binding to the carrier may even take place within the original microbial cell. The enzyme is not influenced in its activity as a result of fixing and may be repeatedly or continuously used fixed to a carrier.

In immobilization by enclosure, the enzyme is generally enclosed between semipermeable membranes and/or gels, microcapsules or fibers. The encapsulated enzymes are separated from the surrounding substrate and product solution, for example, by a semipermeable membrane. Even cells can be encapsulated. The enzyme is not influenced in its activity by fixing in space.

Immobilized enzymes, enzyme-producing microorganisms or cells are used in particular in biotechnological processes. The first industrial processes using immobilized cells were empirically optimized and are still in use today, as for example wastewater treatment in a bacteria bed. Another fairly old process is the production of vinegar by the generator process. In the food industry, the use of cells containing glucose isomerase is the most important process for the production of fructose-containing syrup. Glucose amylase for the production of glucose by the starch process is also used in immobilized form. The splitting of lactose into glucose and galactose using immobilized β-galactosidase from yeasts is another standard process. Other industrial processes using immobilized enzymes are applied in the production of amino acids, in the splitting of penicillin G into 6-aminopenicillic acid and in the production of ethanol with growing immobilized cells of Saccharomyces sp.

Immobilized enzyme and cell systems are used not only in biotechnological production processes, but also in analysis, for example in so-called biosensors. The principle of analysis using immobilized systems is based on the reaction of a substrate to be determined by an immobilized enzyme, the changes in the concentrations of product, substrate and co-substrate being able to be followed, for example by several coupled methods (for example enzyme electrodes).

The disadvantage of the methods known from the prior art for immobilizing enzymes lies in particular in the fact that immobilization of the enzymes has to be carried out in a relatively complicated manner and that combination with systems that are basically inert to the enzymes is not possible.

Now, the problem addressed by the present invention was to provide a process by which biomolecules, more particularly enzymes or enzymatic systems, could be bound even to chemically inert carrier surfaces. More particularly, the invention set out to provide a new process by which biomolecules, more particularly enzymes or enzymatic systems, could be immobilized by binding to chemically inert carrier surfaces.

Another problem addressed by the present invention was to provide a process by which biomolecules, more particularly enzymes or enzymatic systems, could be immobilized in a simple manner, the reactivity of the correspondingly immobilized biomolecules remaining substantially intact, i.e. the correspondingly immobilized biomolecules would not be significantly limited in their reactivity.

Accordingly, the present invention relates to a process for immobilizing biomolecules, more particularly enzymes or enzymatic systems, by fixing or binding to a chemically inert carrier surface, the process comprising the following process steps:

(a) activating the chemically inert carrier surface by modification of that surface by plasmachemical methods and then

(b) binding the biomolecule(s) to be immobilized, optionally after they have been converted into an activated or fixable state, to the carrier surface activated in step (a).

In the step (a) of the process according to the invention, therefore, the chemically inert carrier surface is activated by modification by plasmachemical methods. The plasmachemical modification of surfaces is known per se to the expert (cf. the relevant scientific literature). However, this method has not hitherto been used to prepare surfaces for the binding or immobilization of biomolecules. In other words, the—initially—chemically inert carrier surface is functionalized in step (a) of the process according to the invention.

A “chemically inert carrier surface” in the context of the invention is understood to be a surface which is nonreactive in relation to the particular application. In the present case, this means in particular that, initially, i.e. originally or basically, the carrier surface is not suitable for the binding of biomolecules, i.e. in other words, the carrier surface does not initially have any reactive functional groups which could react with the biomolecule(s) to be fixed or coupled to the surface. Accordingly, “chemically inert” in the context of the present invention means in particular nonreactive to the particular biomolecules or unsuitable for the binding of biomolecules. It is only by the plasmachemical treatment in step (a) of the process according to the invention that the surface of the chemically inert carrier material is prepared for the fixing, i.e. coupling or binding, of the biomolecule(s) or enzyme(s) in subsequent process step (b).

Chemically inert surfaces suitable for the purposes of the invention are any surfaces which do not impair the catalytic activity of the biomolecule, more particularly enzyme, to be fixed or coupled to the surface in process step (b) to any significant extent, if at all, and which do not disrupt enzyme-catalyzed processes to any significant extent, if at all. Such surfaces may be, for example, chemically inert metal surfaces, more particularly surfaces of noble metals or alloys thereof (for example platinum or stainless steel). However, chemically inert plastic surfaces, more particularly surfaces comprising polyhalogenated polymers, preferably polyalkyls or polyalkylenes, such as polytetrafluoroethylene (Teflon®) or polyvinyl chloride (PVC), are also suitable for the purposes of the invention. In addition, any of the usual polymers used for the production of reactors, for example even the already mentioned polyhalogenated polymers, such as polytetrafluoroethylene (Teflon®) or PVC, are suitable as chemically inert surfaces for the binding of biomolecules or enzymes. It is also possible to combine different materials, for example to coat chemically resistant metal surfaces (for example stainless steel or platinum) with polytetrafluoroethylene (Teflon®) or PVC, for example by vapor deposition. Another material suitable for the binding of biomolecules is, for example, cellulose acetate.

The activation of the chemically inert carrier surface in step (a) of the process according to the invention is carried out in particular by directly arranging or fixing at least one suitable functional group reactive to the biomolecules to be bound to the surface under plasmachemical conditions. This method is known per se to the expert. More particularly, the activation of the chemically inert carrier surface in step (a) of the process according to the invention may be carried out in a reactive plasma, more particularly a high-frequency plasma. This is done, for example, in a reactive plasma, more particularly high-frequency plasma, of inert gas(es), such as noble gases for example, and reactant gas(es), such as ammonia for example.

The words “suitable functional group reactive to the biomolecule or enzyme to be bound to the surface” apply to a functional group which is suitable for the direct binding or coupling or for the indirect binding or coupling of the particular biomolecule, more particularly enzyme, i.e. is not reactive to the particular biomolecule or enzyme or reacts with it and, in the process, binds it or couples it to the surface.

After the plasmachemical activation or modification in step (a) of the process according to the invention, the originally chemically inert surface has functional groups which are reactive to the biomolecules to be bound or coupled to the surface.

Non-limiting examples of reactive functional groups suitable for the purposes of the invention are, in particular, groups or groupings which comprise or represent a carboxyl group, an amino group, a hydroxy group and/or a thio group, optionally in protonated or deprotonated form.

The plasmachemical amino, hydroxy and/or carboxyl modification of chemically inert surfaces, more particularly chemically inert surfaces of polytetrafluoroethylene (for example Teflon® membrane) or PVC, is particularly preferred. This method is known per se to the expert.

The particular feature of the plasmachemical activation in step (a) of the process according to the invention is above all the fact that the chemically inert carrier surface is selectively activated solely at the surface. In other words, the bulk properties of the originally chemically inert carrier surface, for example hydrophobic or hydrophilic properties, permeabilities, microporosities, mechanical properties, such as hardness, brittleness, etc., remain otherwise intact during the activation of the chemically inert carrier surface by plasmachemical methods in step (a) of the process according to the invention.

After process step (a), the biomolecule(s) to be immobilized are bound or coupled to the plasmachemically modified carrier surface in process step (b) by binding or coupling of the biomolecules via the functional groups reactive in step (a). The biomolecules may be bound to the reactive functional groups applied to the carrier surface either directly or indirectly, for example via suitable linkers or linker molecules. Corresponding methods are known per se to the expert.

In the context of the invention, a linker—also known as a linker molecule—is understood to be a molecule or part of a molecule which is used to link fragments and/or other molecules (here: linking or joining of plasmachemically activated or modified, chemically inert carrier surfaces to biomolecules, more particularly enzymes).

Before the biomolecule, more particularly enzyme or enzymatic system, is bound or coupled to the plasmachemically activated or modified carrier surface, it may optionally be converted into an activated or fixable state. Corresponding methods are known per se to the expert. Alternatively or simultaneously, the reactive functional group(s) fixed to the carrier surface in step (a) of the process according to the invention may optionally be activated also, for example by protonation, deprotonation, etc., depending on the chemical nature of the functional group(s). Such methods are known per se to the expert.

In the process according to the invention, therefore, the biomolecules may be bound directly or indirectly to a plasmachemically activated, chemically inert carrier surface by covalent and/or ionic bonding, preferably covalent bonding, via the reactive functional group(s) applied in step (a). The biomolecule, more particularly enzyme, is immobilized in this way.

Basically, all types of biomolecules, more particularly all types of enzymes, may be immobilized by the process according to the invention. If the biomolecule to be immobilized is an enzyme, it may be selected for example from the group of oxidoreductases, transferases, hydrolases (for example esterases, such as lipases), lyases, isomerases and ligases (synthetases) and mixtures and combinations thereof.

Process step (b) may optionally be followed by process step (c) which comprises crosslinking the enzymes coupled to the chemically inert carrier surface in process step (b). Crosslinking agents known per se to the expert (for example glutardialdehyde) are generally used to bind the enzymes in process step (c).

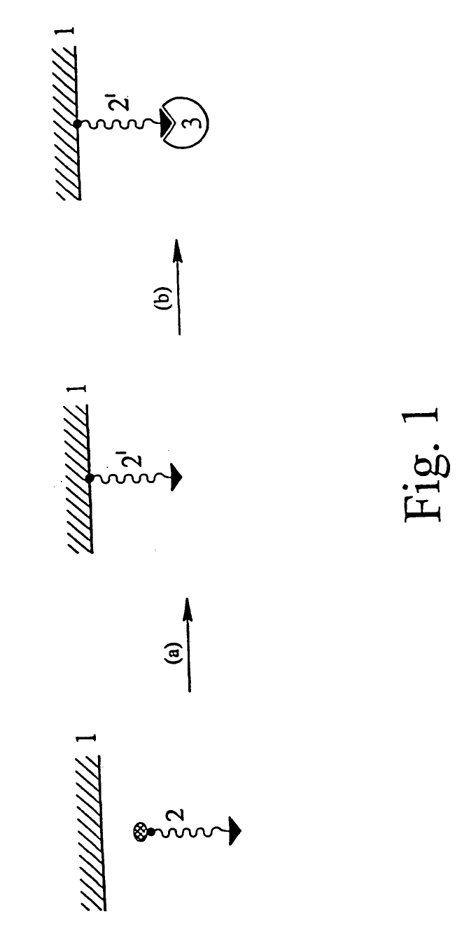

FIG. 1 schematically illustrates the course of the process according to the invention with process steps (a) and (b). First, the activation of the chemically inert carrier surface 1 in process step (a) takes place by modification of the carrier surface 1 by plasmachemical methods, FIG. 1 showing an embodiment in which—starting from a starting molecule 2—a suitable functional group 2′ (for example an amino group) reactive to the biomolecule to be bound to the carrier surface is directly applied to the chemically inert carrier surface 1. As shown in the embodiment illustrated in FIG. 1, the biomolecule 3 to be immobilized is then directly coupled or bound to the carrier surface 1 activated in step (a) in process step (b), the biomolecule 3 being immobilized in this way.

The process according to the invention enables biomolecules, more particularly enzymes, to be bound to chemically inert surfaces and to be immobilized in this way without any effect or significant effect on the reactivity of the biomolecule, more particularly the enzyme. As a result of their immobilization, the biomolecules, more particularly enzymes, can be reused. After use, the enzymes can easily be removed again. In this way, they can be used in particular in high local concentrations and, for example, in continuous throughflow. The substrate specificity and the specificity of the reaction and also the reactivity of the biomolecules, more particularly enzymes, remain intact where immobilization is carried out in accordance with the invention.

The present invention also relates to immobilized and optionally crosslinked biomolecules, more particularly enzymes or enzymatic systems, obtainable by the process according to the invention. The immobilized biomolecules, more particularly enzymes or enzymatic systems, are directly or indirectly fixed, more particularly bound or coupled, to a chemically inert carrier surface, the biomolecule being bound or coupled to the chemically inert carrier surface via suitable reactive functional groups applied to the surface, for example directly via ionic and/or covalent bonds or indirectly via a suitable linker containing biomolecule- or enzyme-reactive functional groups.

The immobilized biomolecules, more particularly enzymes or enzymatic systems, obtainable by the process according to the invention may be used, for example, in biosensors or bioreactors. They may also be used in chromatographic systems, more particularly chromatographic columns, either for preparative purposes or synthesis purposes or even for analytical purposes.

Accordingly, the present invention also relates to biosensors, bioreactors and chromatographic systems comprising the immobilized biomolecules, more particularly enzymes or enzymatic systems, obtainable in accordance with the invention.

As described above, the immobilized biomolecules, more particularly enzymes or enzymatic systems, obtainable by the process according to the invention may also be used in biosensors. “Biosensors” in the context of the invention are understood to be sensors comprising a bioactive component based on the coupling of biomolecules which, as receptors in the broadest sense, specifically recognize analytes and physicochemical transductors which convert a biologically generated signal (for example oxygen concentration, pH value, dye, etc.) into electrical signals.

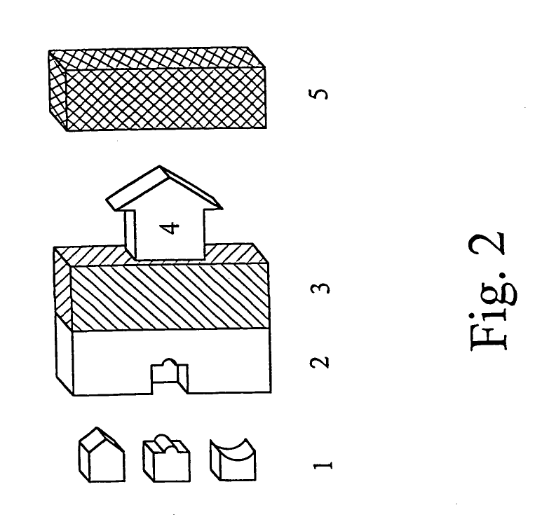

FIG. 2 illustrates the typical structure of a biosensor for specifically recognizing an analyte 1, the biosensor comprising a receptor 2 and a transductor 3 which converts the biological signal generated by the receptor 2 into an electronic signal 4 which is transmitted to an electronic circuit 5.

Various biomolecules, more particularly enzymes, may be used for specific recognition. The transductors used may be potentiometric sensors, amperometric electrodes, piezoelectric sensors, thermistors or optoelectronic sensors. There are in particular two basic types of biosensors, depending on the reaction or interaction of the analyte with the receptor. On the one hand, there are bioaffinity sensors, which use the change in electron density occurring during complexing, and on the other hand metabolism sensors which are based on the specific recognition and reaction of substrates.

Biosensors are used—particularly in the form of enzyme electrodes—in healthcare, for monitoring biotechnological processes, in the food industry or in environmental protection. Various systems, for example glucose, galactose, lactose, ethanol, lactic acid or uric acid, can be analyzed with biosensors.

Where the immobilized enzymes are used in conventional biosensors, the enzyme molecules are introduced either into polymeric matrixes (for example PVC, gels, graphites or zeolites) or between films (for example cellulose acetate). By contrast, the concept according to the invention is characterized in that, in sensors which are based, for example, on the enzymatic production or the consumption or oxygen and which have a chemically inert membrane (for example a Teflon® membrane), the membrane is used to bind enzymes by the process according to the invention. As described above, biomolecule- or enzyme-reactive functional groups suitable for this purpose, for example amino or carboxyl groups, have to be bound to the chemically inert membrane surface. These groups may be applied by a plasma coating process. Crosslinking agents may then optionally be used to bind the immobilized enzymes. Examples of suitable biosensor systems according to the invention are, for example, catalase and/or glucoseoxidase in whose case coupling of the particular enzyme onto a suitable functional group bound to a chemically inert surface may be followed by crosslinking with glutardialdehyde (for example 5% in buffer, pH 7 and 20 mg catalase (Merck) or glucoseoxidase (Merck) to 500 pi solution). Other examples of embodiment comprise the binding of 1,000 to 100,000 U (units) catalase or 10 to 100 U glucoseoxidase by difunctional glutardialdehyde to aminated PTFE membranes with a membrane diameter of 1 to 10 mm, preferably 8 mm. However, the ranges mentioned do not represent limitations, but rather have to be co-ordinated with the particular type of application taking the desired concentration range of the analyte to be measured into consideration. Oxygen-sensitive enzymatic sensors such as these have a life of around 2 months.

The biosensors according to the invention are suitable, for example, for the production of microelectrode (arrays) for small volumes and high sample throughputs (for example for combinatorial use).

Reference has already been made to the combined immobilization of the two enzymes catalase and glucoseoxidase. This is of analytically relevant importance when, for example, measured values relating to glucose have to be continuously monitored in media low in oxygen or even free from oxygen. In addition, the process described in the following enables the measuring range to be extended.

However, since oxygen is an important reactant in the substrate reaction of glucoseoxidase (GOD):

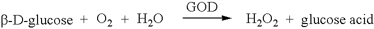

the solution to the problem lies in the simultaneous supply of chemically bound oxygen which then still has to be released within the enzyme membrane for the substrate reaction of the β-D-glucose by GOD. This can be done in a particularly elegant manner on the basis of a bienzyme membrane if, for example, not only glucoseoxidase but also catalase is covalently bonded by glutardialdehyde and crosslinked in accordance with the invention on an aminated PTFE membrane. The two enzymes may be immobilized in mixed form and also in layers. The layered immobilization may be carried out in two or more layers. The sequence of the enzyme layers can, but need not, be of application-influenced importance.

If, now, in an oxygen-sensitive enzymatic throughflow sensor comprising the described bienzyme membrane in its embodiment according to the invention, not only β-D-glucose—which is in equilibrium with the α-form where the mutarotation equilibrium is adjusted—but at the same time chemically bound oxygen in the form of H₂O₂ surges in, catalase will make oxygen available from hydrogen peroxide for the substrate reaction of the glucoseoxidase in accordance with the following reaction equation:

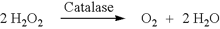

Since, on the one hand, the membrane electrodes are oxygen-sensitive enzymatic membrane electrodes and since, on the other hand, oxygen is one of the reactants of the glucoseoxidase, the hydrogen peroxide should be supplied in a constant concentration. In addition, in the case of an oxygen-containing measuring medium, provision should also be made for the physically dissolved oxygen to reach the sensor in a constant concentration. Accordingly, one particular embodiment of the invention is a biosensor, more particularly for measuring glucose, preferably β-D-glucose, the biosensor comprising at least one peroxide-sensitive biomolecule, more particularly enzyme, preferably glucoseoxidase, optionally in combination with catalase, fixed to an activated, plasmachemically modified and/or functionalized carrier surface of a chemically inert carrier material, preferably polytetrafluoroethylene.

In another particular embodiment of the present invention, the biosensor is a peroxide sensor or a biochemical peroxide sensor. In the context of the invention, such a sensor is a biosensor or a measuring element which reacts qualitatively or quantitatively, or is sensitive, to the presence or concentration of inorganic or organic peroxides (for example hydrogen peroxide, dissolved peroxides from inorganic or organic salts, organic peracids, etc.). A (biochemical) peroxide sensor such as this contains—almost as an active component—molecules which indicate the presence of, or react with, peroxides. These molecules may be, for example, molecules, more particularly hydrogen-peroxide-sensitive enzymes, which are fixed by binding to an activated, chemically inert carrier surface without any basic change in their reactivity.

In a preferred embodiment of the invention, the biochemical peroxide sensor according to the invention is a biosensor for the qualitative and/or quantitative determination of organic or inorganic peroxides, more particularly hydrogen peroxide; dissolved peroxides from inorganic or organic salts; and/or organic peracids, the biochemical peroxide sensor comprising at least one peroxide-sensitive biomolecule, more particularly enzyme, preferably catalase, fixed or bound to an activated, plasmachemically modified or functionalized carrier surface of a chemically inert carrier material, more particularly polytetrafluoroethylene (for example Teflon® membrane). In other words, one or more biomolecules or enzymes complementing one another in their effect may be fixed to or arranged on the carrier either directly or indirectly via linkers: the enzyme actually reacting with hydrogen peroxide can be a catalase for example. Such enzymes are commercially available, for example from Merck KGaA in Germany. The biomolecules or enzymes bound to the chemically inert carrier surface may then be crosslinked, for example with glutardialdehyde. The biomolecules or enzymes are fixed or bound to the surface by suitable reactive functional groups applied to the carrier surface beforehand by plasmachemical methods known per se.

In one particularly preferred embodiment, the carrier surface consists of plasmachemically surface-modified polytetrafluoroethylene (Teflon®) or has a coating thereof (for example a metal, for example platinum, coated with polytetrafluoroethylene by vapor deposition), the surface of the Teflon® layer being modified as described above and the enzyme(s) being fixed thereto. The surface modification of the Teflon preferably comprises plasmachemically producing or arranging chemically reactive groups, for example amino or carboxyl groups, on the carrier surface and then binding the enzyme(s) thereto, optionally followed by crosslinking. In the biochemical peroxide sensor according to the invention, the unit of biomolecule(s), more particularly enzyme(s), and the carrier surface supplies a preferably electrical signal from which the presence and/or concentration of peroxides can be deduced (for example on the basis of a previously established calibration curve).

Accordingly, the biochemical peroxide sensor according to the invention enables peroxides to be qualitatively and/or quantitatively determined in free or bound form, for example in the form of free hydrogen peroxide, in the form of peracids, in the form of perborates or in the form of soluble peroxides. In all these cases, a certain concentration of the per compound is in equilibrium with a certain concentration of hydrogen peroxide which can be determined by the peroxide sensor according to the invention. For example, the level of the electrical signal of the peroxide sensor according to the invention can be correlated with the concentration of the particular per compound present by corresponding calibration curves. If it is desired to determine the concentration of hydrogen peroxide in free or bound form, the measuring solution is preferably buffered to a pH of 4.5 to 8.

The presence or concentrations of molecules which react with the peroxides or cause peroxide to be consumed (for example nitrite or hydroxylamine) can also be indirectly determined with the peroxide sensor according to the invention. The reaction of the peroxide-consuming molecules with the peroxides (for example hydrogen peroxide) or the competitive reaction of the peroxide sensor is utilized for this purpose. For example, a known quantity of hydrogen peroxide may be added to the solution of the peroxide-consuming molecules, the level of the electrical signal of the biochemical peroxide sensor may be measured and compared with the theoretically expected level (for example on the basis of calibration) and the concentration of the peroxide-consuming molecules derived from the difference.

The peroxide sensor according to the invention is suitable, for example, for process monitoring and process control (for example in phosphating).

Accordingly, where the biomolecules, more particularly enzymes or enzymatic systems, immobilized in accordance with the invention are used in biosensors, at least two different types of biomolecules, more particularly enzymes or enzymatic systems, can be combined with one another, i.e. in particular so-called enzyme chains or enzyme degradation chains may be used. The various enzymes may either be present in one and the same reaction system (for example in a measuring cell) or may be sequentially arranged “in series” (for example in successive measuring cells). However, a multiple measuring chain structure arranged in parallel may also be of advantage (for example for differential measurements). In this way, for example, several substances can be measured in parallel by several enzymes (or enzyme chains) in one and the same measuring cell or in measuring systems. Reference may be made in this regard to patent application DE 101 18 554.5 of which the entire disclosure is hereby included by reference in the present application.

As described above, the immobilized biomolecules, more particularly enzymes or enzymatic systems, obtainable by the process according to the invention may also be used in bioreactors.

In the context of the invention, “bioreactors” are understood to be the physical container in which biological conversions are carried out, more particularly with biomolecules, such as enzymes.

The process according to the invention may be used, for example, to modify the wall surfaces of bioreactors. The bioreactors may be of any type, for example reactors with planar surfaces and also tubular reactors. Examples of such reactors are polytetrafluoroethylene-coated enzyme reactors to the walls of which the enzyme is bound by the above-described pretreatment according to the invention so that a more efficient generation of reactors in which the enzymes do not have to be removed from the reaction solution can be created. Reactor surfaces suitable for the purposes of the invention may consist, for example, of metal or may be coated with any of the polymers typically used for the production of reactors (for example Teflon® or PVC) or a combination of these materials.

In cases where the biomolecules, more particularly enzymes or enzymatic systems, immobilized in accordance with the invention are used in bioreactors, it is also possible in another embodiment of the present invention, particularly in the case of fixed-bed reactors, to bind the immobilized biomolecule, more particularly enzyme or enzymatic system, to the stationary carrier material or bulk material.

In cases where the biomolecules, more particularly enzymes or enzymatic systems, immobilized in accordance with the invention are used in bioreactors, more particularly for modifying the wall surface or in the binding of the enzyme to the carrier material or bulk material, at least two different types of biomolecules, more particularly enzymes or enzymatic systems, may be combined with one another, i.e. so-called biomolecule or enzyme chains or biomolecule or enzyme degradation chains may be used. The various biomolecules, more particularly enzymes or enzymatic systems, may either be present in a single reaction zone or may be sequentially arranged “in series” (for example in successive reaction zones). For example, multistage, enzyme-catalyzed syntheses and processes can be carried out in this way.

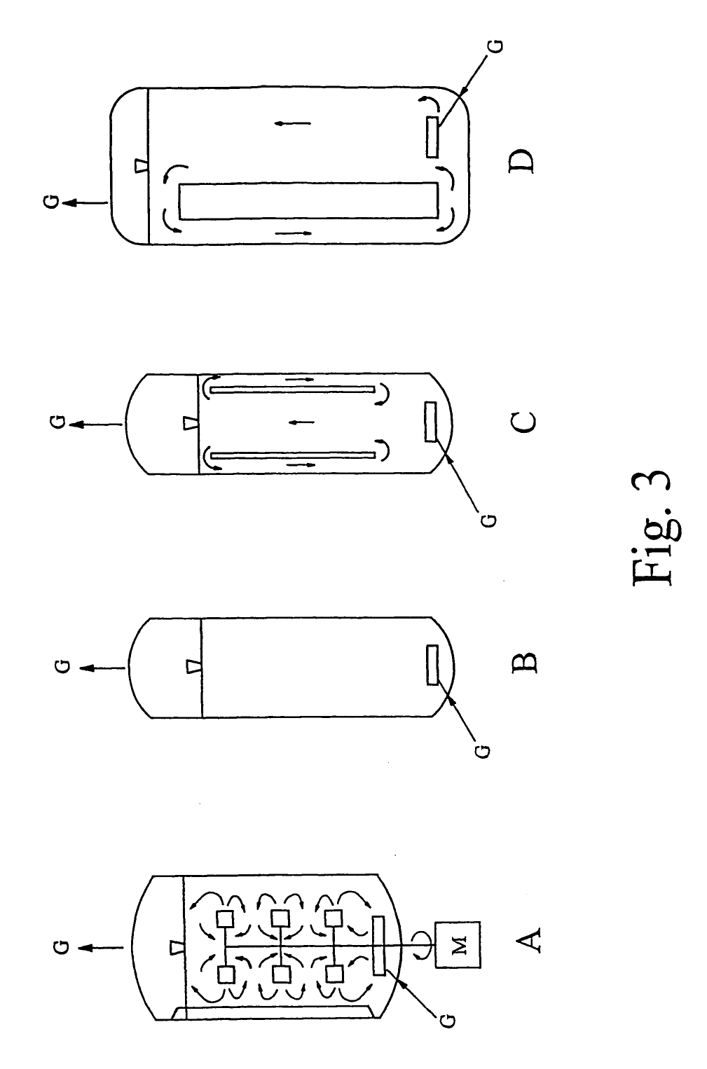

By way of example, FIG. 3 schematically illustrates various types of known bioreactors.

FIG. 3A shows a stirred tank reactor in which energy is introduced by mechanically moved units. The letter G denotes the gas stream and the letter M denotes the mechanical drive system (for example motor). By virtue of their versatility, stirred tank reactors are the most commonly used.

FIG. 3B shows a bubble column reactor where mixing is effected by the introduction of air or another gas. The letter G denotes the gas stream.

FIG. 3C shows a so-called airlift fermenter with internal throughflow, the circulation of liquid and mixing generally being achieved by the introduction of air or another gas. The letter G denotes the gas stream.

FIG. 3D shows a so-called airlift fermenter with external throughflow, the circulation of liquid and mixing generally being achieved by the introduction of air or another gas. The letter G denotes the gas stream.

The above-described types of known bioreactors may be modified in accordance with the invention, for example by modification of the wall surface (for example by the coupling of biomolecules, more particularly enzymes or enzymatic systems, onto the chemically inert reactor walls in accordance with the invention) or—in the case of fixed-bed bioreactors—by binding of the biomolecules, more particularly enzymes or enzymatic systems, to the carrier material or bulk material.

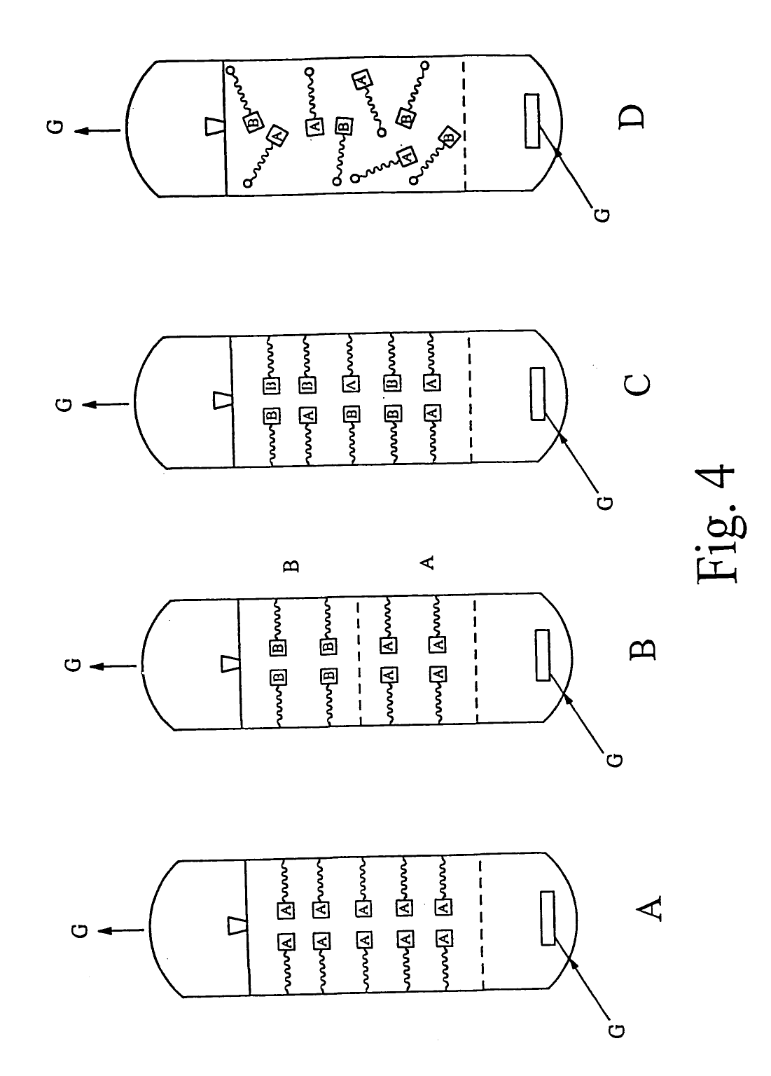

By way of example, FIG. 4 schematically illustrates some embodiments of bioreactors according to the invention.

FIG. 4A shows a bioreactor of which the chemically inert walls are modified by coupling of an immobilized biomolecule, more particularly enzyme, of type A. The letter G denotes the gas stream.

FIG. 4B shows a bioreactor of which the chemically inert walls are modified by coupling of an immobilized biomolecule, more particularly enzyme, of type A and an immobilized biomolecule, more particularly enzyme, of type B which are arranged in different successive reaction zones. The letter G denotes the gas stream.

FIG. 4C shows a bioreactor of which the chemically inert walls are modified by coupling of an immobilized biomolecule, more particularly enzyme, of type A and an immobilized biomolecule, more particularly enzyme, of type B which are arranged in a single reaction zone.

FIG. 4D shows a bioreactor in the form of a fixed-bed reactor onto whose carrier material or bulk material immobilized biomolecules, more particularly enzymes, of type A and type B arranged in a single reaction zone are coupled.

There are many other variants for the modification of bioreactors, more particularly bioreactor wall surfaces and/or bioreactor bulk material and the like, in accordance with the invention which the expert will readily consider on reading the present specification without departing from the scope of the invention.

The biomolecules, more particularly enzymes or enzymatic systems, immobilized in accordance with the invention may also be used in chromatographic systems, more particularly in chromatographic columns. This may be done for preparative or synthesis purposes (for example carrying out enzyme-catalyzed reactions in a chromatographic column) or even for analysis purposes (for example in analytical column chromatography).

The use of the biomolecules, more particularly enzymes or enzymatic systems, immobilized in accordance with the invention in biosensors, bioreactors and chromatographic systems has the advantage that, on the one hand, the biomolecules, more particularly enzymes or enzymatic systems, can be reused as a result of immobilization and, on the other hand, can easily be removed after use (for example after synthesis in the bioreactor,, for example by draining off the reaction mixture). In this way, the biomolecules, more particularly enzymes or enzymatic systems, can be efficiently and inexpensively used in a high local concentration and in continuous throughflow. However, the substrate specificity and the specificity of the reaction and also the reactivity of the enzymes are not lost as a result of immobilization in accordance with the invention.

The object of the process according to the invention is inter alia to apply the chemically inert surface material (for example Teflon®) as a diffusion barrier by sputtering—in a plasma—and then directly to apply the functional groups or, solely for reactor applications, the amino or carboxyl groups onto which the biomolecules, more particularly enzymes or enzymatic systems, may then be coupled.

Other embodiments and variations of the present invention will be readily apparent and practicable to the expert on reading the present specification without departing from the scope of the invention.

The following Examples are intended to illustrate the invention without limiting it in any way.

### EXAMPLES

### Production of Bioelectrochemical Peroxide and Glucose Sensors according to the Invention

A chemically inert carrier surface, in the present case a PTFE membrane (Teflon® membrane) is first functionalized in a reactive high-frequency plasma under conditions known per se. In the process, suitable enzyme-reactive functional groups, more particularly amino and/or carboxyl groups, are bound to the chemically inert membrane surface.

After they have been punched out, 13 mm diameter aminated PTFE membranes are clamped by an O ring onto an acrylic glass ring so that an 8 mm diameter flat surface is formed onto which enzymes can be covalently bonded and crosslinked on the measuring solution side, for example according to the particular application (measuring range):

for various H₂O₂ sensors: 1,000 to 100,000 U catalase/membrane

for various glucose sensors: 10 to 100 U glucoseoxidase/membrane.

On the detector side, the cavity of the acrylic glass ring is charged with an internal electrolyte for connecting the measuring cathode of Pt and the Ag/AgCl reference anode.

After the acrylic glass ring with the membrane system has been pushed onto an oxygen electrode and locked in a throughflow chamber, bioelectrochemical sensors according to the invention of the type defined above in the general description are obtained. These sensors have a life of about two months.

Strictly speaking, the glucose sensors according to the invention measure β-D-glucose. However, since the β- and α-forms of glucose are present in a constant ratio after establishment of the mutarotation equilibrium, the sensors may be called glucose sensors in the present case because the calibration process also takes these factors into account.

For example, the following biosensors according to the invention were produced:

1. Glucose sensor with the laboratory code 2.) SBC-1320-β-D-glucose-HDKS-No.1: 40 U glucoseoxidase covalently bonded by glutardialdehyde to the carboxylated PTFE membrane of an oxygen detector.

2. Glucose sensor with the laboratory code 3.) SBC-1321-β-D-glucose-HDKS-No.1: 40 U glucoseoxidase covalently bonded by glutardialdehyde to the aminated PTFE membrane of an oxygen detector.

3. Hydrogen peroxide sensor with the laboratory code 5.) SBC-1323-H₂O₂-HDKS1-No.1: 26,000 U catalase covalently bonded by glutardialdehyde to the aminated PTFE membrane of an oxygen detector.

The glucose sensor with the laboratory code 3.) SBC-1321-β-D-glucose-HDKS-No.1 is a membrane system based on an aminated PTFE membrane with GOD covalently bonded by glutardialdehyde, the amination of the PTFE membrane and the subsequent immobilization of the GOD being carried out by the process according to the invention. This membrane system was integrated into an amperometric throughflow cell with a Pt measuring cathode and an Ag/AgCl reference anode, resulting in the formation of an oxygen-sensitive enzymatic β-D-glucose sensor for throughflow measurements. The sensor system according to the invention was tested in an endurance test. To this end, the sensor according to the invention was subjected to continuous perfusion with a phosphate buffer (pH 7.04 at 25° C.). Over this period, around 100 l of the buffer were pumped through the measuring system. Finally, more glucose measurements were carried out with the phosphate buffer as solvent for, the analyte for demonstrating the serviceability of the membrane system (pump: Permax 12/6 at 20 digits with 1.9×4.5 mm silicone tube). Despite 1 year's continuous perfusion of the sensor according to the invention with phosphate buffer (HPL) at a temperature of 20 to 25° C., the enzymatic activity of the membrane system can still be regarded as adequate even after one year.

Biosensors containing glucoseoxidase and catalase were also produced. Biosensors such as these may be used, for example, for continuously monitoring the measured values relating to glucose in media low in oxygen or even free from oxygen. In addition, this allows the measuring range to be extended. The concentrations of catalase or glucoseoxidase units (U) to be immobilized are shown below for these biosensors. The two above-mentioned enzymes may also be unconditionally present together in immobilized form in the membrane system. This is demonstrated by three examples showing the membrane composition, the covalent bonding and crosslinking of the two enzymes on an aminated PTFE membrane again being effected by glutardialdehyde:

| | Catalase | Glucoseoxidase |
|---|---|---|
| I. | 2.6 × 10⁴ U | 40 U |
| II. | 5.2 × 10⁴ U | 80 U |
| III. | 7.8 × 10⁴ U | 120 U |

In the bioelectrochemical througflow sensors according to the invention, the measuring medium can be taken in by a following roller pump.

1. A process for immobilizing biomolecules, more particularly enzymes or enzymatic systems, by fixing or binding to a chemically inert carrier surface, the process comprising the following process steps:
(a) activating the chemically inert carrier surface by modification of that surface by plasmachemical methods and then
(b) binding at least one of the biomolecules to be immobilized, optionally after it has been converted into an activated, fixable state, to the carrier surface activated in step (a).

2. A process as claimed in claim 1, characterized in that the activation of the chemically inert carrier surface in step (a) comprises functionalizing the chemically inert carrier surface.

3. A process as claimed in claim 1 or 2, characterized in that the activation of the chemically inert carrier surface in step (a) is carried out by applying at least one suitable functional group reactive to the biomolecules to be bound to the chemically inert carrier surface directly to that surface under plasmachemical conditions.

4. A process as claimed in any of the preceding claims, characterized in that the reactive functional group is a carboxyl, amino, hydroxy and/or thio group, optionally in activated, more particularly protonated or deprotonated form,

5. A process as claimed in any of the preceding claims, characterized in that the activation of the chemically inert carrier surface by plasmachemical methods in step (a) is selectively carried out on the surface only.

6. A process as claimed in any of the preceding claims, characterized in that, during the activation of the chemically inert carrier surface by plasmachemical methods in step (a), the bulk properties of that surface remain otherwise intact.

7. A process as claimed in any of the preceding claims, characterized in that the activation of the chemically inert carrier surface in step (a) is carried out in a reactive plasma, more particularly a high-frequency plasma.

8. A process as claimed in any of the preceding claims, characterized in that the chemically inert carrier surface comprises noble metals, such as in particular platinum and alloys thereof, stainless steel or polyhalogenated polymers, more particularly polyhalogenated polymeric hydrocarbons, such as in particular polytetrafluoroethylene or polyvinyl chloride, or cellulose acetate or combinations of these materials.

9. A process as claimed in any of the preceding claims, characterized in that, in step (b), the binding or coupling of the biomolecules to be immobilized onto the plasmachemically modified carrier surface is carried out by binding or coupling of the biomolecules via the functional groups reactive in step (a).

10. A process as claimed in claim 9, characterized in that, in step (b), the biomolecules are bound to the reactive functional groups applied to the surface either directly or indirectly via a suitable linker.

11. A process as claimed in any of the preceding claims, characterized in that the biomolecules are directly or indirectly bound to the carrier surface by covalent and/or ionic bonding, preferably covalent bonding, via the reactive functional group(s).

12. A process as claimed in any of the preceding claims, characterized in that the biomolecule to be immobilized is an enzyme and is selected in particular from the group consisting of oxidoreductases, transferases, hydrolases such as, in particular, esterases such as lipases, lyases, isomerases and ligases (synthetases) and mixtures or combinations thereof.

13. A process as claimed in any of the preceding claims, characterized in that process step (b) may be followed by a process step (c) comprising crosslinking of the biomolecules bound or coupled to the chemically inert carrier surface in process step (b), process steps (b) and (c) optionally being carried out in combination and, more particularly, at the same time.

14. An immobilized biomolecule, more particularly enzyme or enzymatic system, obtainable by the process claimed in claims 1 to 13.

15. An immobilized biomolecule, more particularly enzyme or enzymatic system, characterized in that the biomolecule is directly or indirectly fixed, more particularly bound or coupled, to a chemically inert carrier surface, the biomolecule being bound or coupled to the chemically inert carrier surface via suitable reactive functional groups applied to that surface.

16. The use of the immobilized biomolecule, more particularly enzyme or enzymatic system, claimed in claim 14 or 15 in bioreactors or biosensors.

17. The use claimed in claim 16 for modifying the wall surfaces of bioreactors.

18. The use claimed in claim 16, characterized in that the immobilized biomolecule, more particularly enzyme or enzymatic system, is bound to the carrier material or bulk material of a bioreactor.

19. The use of the immobilized biomolecule, more particularly enzyme or enzymatic system, claimed in claim 14 or 15 in chromatographic systems, more particularly chromatographic columns.

20. The use claimed in claim 19 for analysis or preparation/synthesis purposes.

21. Biosensors, bioreactors or chromatographic systems containing the immobilized biomolecule, more particularly enzyme or enzymatic system, claimed in claim 14 or 15.

22. A biosensor, more particularly for the qualitative and/or quantitative determination of peroxides, more particularly organic and/or inorganic peroxides, such as hydrogen peroxide; dissolved peroxides from inorganic or organic salts; and/or organic peracids, the biosensor comprising at least one peroxide-sensitive biomolecule, more particularly enzyme, preferably catalase, fixed to an activated, plasmachemically modified and/or functionalized carrier surface of a chemically inert carrier material, preferably polytetrafluoroethylene.

23. A biosensor, more particularly for the qualitative and/or quantitative determination of glucose, more particularly β-D-glucose, the biosensor comprising at least one peroxide-sensitive biomolecule, more particularly enzyme, preferably glucoseoxidase, optionally in combination with catalase, fixed to an activated, plasmachemically modified and/or functionalized carrier surface of a chemically inert carrier material, preferably polytetrafluoroethylene.

24. A biosensor as claimed in claim 22 or 23, characterized in that the biomolecules, more particularly enzymes, bound to the chemically inert carrier surface are additionally crosslinked, more particularly with glutardialdehyde.

25. A biosensor as claimed in any of claims 22 to 24, characterized in that, in the biosensor, the unit of biomolecule(s), more particularly enzyme(s), and the carrier surface supplies a preferably electrical signal, the presence and/or concentration of peroxides or glucose, more particularly β-D-glucose, being deducible in particular on the basis of that signal.

26. Biosensors, more particularly as claimed in any of claims 21 to 25, containing glucoseoxidase and/or catalase in immobilized form.
